(19) United States
(12) Patent Application Publication
(10) Pub. No.: US 2003/0190268 A1
(43) Pub. Date: Oct. 9, 2003

(54) Single deodorizer for attaching to a variety of fans without modification

(76) Inventor: Michelle Saverd, South Plainfield, NJ (US)

Correspondence Address: Michelle Saverd, 217 WEST CRESENT PARKWAY, South Plainfield, NJ, 07080, US

(21) Appl. No.: 10/118,509

(22) Filed: Apr. 5, 2002

(51) Int. Cl. A61L 9/12

(52) U.S. Cl. 422/124

(57) ABSTRACT

A single deodorizer that attaches to a variety of fans without modification. The variety of fans include a table fan that has blades and a cage that encases the blades and a ceiling fan that has blades with ceiling facing-surfaces. The deodorizer includes a housing, a deodorizing element, attaching apparatus, and selecting apparatus. The housing attaches to the cage of the table fan and to the ceiling facing-surface of at least one blade of the ceiling fan and is circular-cylindrically-shaped for reducing drag thereon when attached to the ceiling facing-surface of the at least one blade of the ceiling fan. The deodorizing element is removably disposed in the housing and selectively generates different amounts of a deodorant through the housing. The attaching apparatus attaches the housing to the cage of the table fan and to the ceiling facing-surface of the at least one blade of the ceiling fan without modification. The selecting apparatus selectively generates the different amounts of the deodorant through the housing.

### BACKGROUND OF THE INVENTION

1. Field of the Invention

The present invention relates to a single deodorizer. More particularly, the present invention relates to a single deodorizer for attaching to a variety of fans without modification.

2. Description of the Prior Art

There are a myriad of odors associated with buildings and residences. Some of these odors, such as cooking odors, can be quite pleasant, whereas, others, such as smoking odors can be quite unpleasant.

Accordingly, there are a variety of room freshening deodorant dispensing devices known in the art. These devices include simple sprays and wick-type dispensers, for example.

While somewhat effective in some circumstances, the deodorant devices similar to the above-mentioned devices have several drawbacks. For example, these devices are somewhat restricted in the amount of area that can be effectively treated, and could be limited to a particular portion of a single room. Furthermore, such devices can create a strong odor gradient that can be quite noticeable, and hence, can be undesirable. An odor gradient has a very strong odor in an area adjacent to the dispenser and a very weak odor in an area spaced from that dispenser.

Other means for dispensing deodorant into a specified area include deodorant obtaining and dispensing filters for use in a central heating and cooling system. Such devices, however, may not be long lasting enough to be economically feasible, especially if the building has many rooms. Some rooms may require deodorizing, while others do not, yet such room selection is not possible using centrally located deodorant dispensing means without cutting off a room from being heated or cooled, which may not be desirable. Furthermore, such devices may be expensive to install and replace.

Other means for dispensing deodorant into a room have included placing deodorant dispensing devices on the blades of a fan. such devices have the advantage of wide dispersant plus room-specific characteristics. These devices have been placed on table fans as well as ceiling fans.

There exists, however, a need for a single deodorizer that can be attached to either a table fan or a ceiling fan without modification.

Numerous innovations for deodorizers for ceiling fans have been provided in the prior art that will be described. Even though these innovations may be suitable for the specific individual purposes to which they address, however, they differ from the present invention in that they do not teach a single deodorizer for attaching to a variety of fans.

FOR EXAMPLE, U.S. Patent Number Des. No. 324,910 to Portis teaches the ornamental design for an air freshener for mounting on a blade of a ceiling fan.

ANOTHER EXAMPLE, U.S. Pat. No. 4,666,670 to Cox teaches a basket containing perfumed potpourri that is suspended beneath a ceiling fan so that air from the fan is circulated through the basket and perfumed by the potpourri. The basket has a bottom portion of relatively tightly formed mesh for retaining the potpourri and an upper portion of relatively loosely formed mesh for providing air circulation through the basket.

STILL ANOTHER EXAMPLE, U.S. Pat. No. 4,944,898 to Glaser teaches an air freshener dispensing device that is mounted on the blade of a ceiling fan and includes channels that are shaped to increase the velocity of air relative to the air freshener product over the velocity of the air relative to the case in which the air freshener product is contained as the fan blade rotates. The case is releasably mounted on the fan blade so the product can be changed or replenished.

YET ANOTHER EXAMPLE, U.S. Pat. No. 5,383,765 to Baxter et al. teaches an air freshener apparatus having a recess defined by a tray, two sidewalls extending upwardly therefrom with overlapping flanges, and an upstanding end wall slidably received on a blade of a ceiling fan with a foam pad impregnated with a scent material received in the recess. Two hook-like arms extend outwardly from the tray for slidingly holding the air freshener apparatus on an upper surface of the fan blade.

STILL YET ANOTHER EXAMPLE, U.S. Pat. No. 5,775,876 to Walker et al. teaches a ceiling-fan-blade-mounted air freshener apparatus that includes a housing assembly which includes a lower housing portion and an upper housing portion projecting upward from the lower housing portion. The upper housing portion includes a plurality of vent apertures. An air freshener/matrix assembly is housed within the housing assembly. A housing-to-blade connector connects the housing assembly to a ceiling fan blade. In one embodiment, the housing-to-blade connector is comprised of a layer of pressure-sensitive adhesive bonded to a bottom side of the lower housing portion. The housing-to-blade connector further includes a peel strip removably adhered to a bottom surface of the layer of pressure-sensitive adhesive. The housing assembly includes a pointed end, and the air freshener/matrix assembly includes a complementary pointed end. The pointed end of the air freshener/matrix assembly is wedged into the pointed end of the housing assembly under an influence of centrifugal force. In a second embodiment, the housing-to-blade connector is comprised of a clip assembly which includes a pair of spring clamp members which project upward from a clip base member. A centrifugal stop member projects upward from the clip base member. A layer of pressure-sensitive adhesive is bonded to a bottom side of the clip base member. A peel strip is removably adhered to a bottom surface of the layer of pressure-sensitive adhesive. A carrier assembly retains the air freshener/matrix assembly. The carrier assembly includes a pointed end.

It is apparent that numerous innovations for deodorizers for ceiling fans have been provided in the prior art that are adapted to be used. Furthermore, even though these innovations may be suitable for the specific individual purposes to which they address, however, they would not be suitable for the purposes of the present invention as heretofore described.

### SUMMARY OF THE INVENTION

ACCORDINGLY, AN OBJECT of the present invention is to provide a single deodorizer for attaching to a variety of fans without modification that avoids the disadvantages of the prior art.

ANOTHER OBJECT of the present invention is to provide a single deodorizer for attaching to a variety of fans without modification that is simple and inexpensive to manufacture.

STILL ANOTHER OBJECT of the present invention is to provide a single deodorizer for attaching to a variety of fans without modification that is simple to use.

BRIEFLY STATED, YET ANOTHER OBJECT of the present invention is to provide a single deodorizer that attaches to a variety of fans without modification. The variety of fans include a table fan that has blades and a cage that encases the blades and a ceiling fan that has blades with ceiling facing-surfaces. The deodorizer includes a housing, a deodorizing element, attaching apparatus, and selecting apparatus. The housing attaches to the cage of the table fan and to the ceiling facing-surface of at least one blade of the ceiling fan and is circular-cylindrically-shaped for reducing drag thereon when attached to the ceiling facing-surface of the at least one blade of the ceiling fan. The deodorizing element is removably disposed in the housing and selectively generates different amounts of a deodorant through the housing. The attaching apparatus attaches the housing to the cage of the table fan and to the ceiling facing-surface of the at least one blade of the ceiling fan without modification. The selecting apparatus selectively generates the different amounts of the deodorant through the housing.

The novel features which are considered characteristic of the present invention are set forth in the appended claims. The invention itself, however, both as to its construction and its method of operation, together with additional objects and advantages thereof, will be best understood from the following description of the specific embodiments when read and understood in connection with the accompanying drawing.

### BRIEF DESCRIPTION OF THE DRAWING

The figures of the drawing are briefly described as follows:

FIG. 1 is a diagrammatic perspective view of the present invention utilized with a table fan;

FIG. 2 is a diagrammatic perspective view of the present invention utilized with a ceiling fan;

FIG. 3 is an enlarged diagrammatic perspective view of the area generally enclosed in the dotted curve identified by ARROW 3 in FIGS. 1 and 2 of the present invention;

FIG. 4 is a diagrammatic side elevational view taken generally in the direction of ARROW 4 in FIG. 3; and

FIG. 5 is an enlarged diagrammatic cross sectional view taken on LINE 5-5 in FIG. 3.

### LIST OF REFERENCE NUMERALS UTILIZED IN THE DRAWING

| | |
|---|---|
| 10 | single deodorizer of the present invention for attaching to |
| | variety of fans without modification |
| 12 | table fan |
| 14 | blades of table fan 12 |
| 16 | meshed cage of table fan 12 |
| 18 | ceiling fan |
| 20 | blades of ceiling fan 18 |
| 22 | ceiling facing-surfaces of blades 20 of ceiling fan 18 |
| 24 | housing for attaching to cage 16 of table fan 12 and to |
| | ceiling-facing surface 22 of at least one blade 20 of ceiling |
| | fan 18 |
| 26 | deodorizing element |
| 28 | first apparatus for attaching housing 24 to cage 16 of table |
| | fan 12 and to ceiling facing-surface 22 of at least one blade |
| | 20 of ceiling fan 18 without modification |
| 30 | second apparatus for selectively generating different amounts |
| | of deodorant through housing 24 |
| 32 | fixed portion of housing 24 for replaceably attaching to cage |
| | 16 of table fan 12 and to ceiling-facing surface 22 of at |
| | least one blade 20 of ceiling fan 18 |
| 34 | replaceable portion of housing 24 |
| 36 | center of replaceable portion 34 of housing 24 |
| 38 | rear wall of fixed portion 32 of housing 24 |
| 40 | center of rear wall 38 of fixed portion 32 of housing 24 |
| 42 | fan-facing surface of rear wall 38 of fixed portion 32 of |
| | housing 24 |
| 44 | fan-opposing surface of rear wall 38 of fixed portion 32 of |
| | housing 24 |
| 46 | front wall of fixed portion 32 of housing 24 |
| 48 | plurality of first throughvents in rear wall 38 of fixed |
| | portion 32 of housing 24 |
| 50 | throughbore in replaceable portion 34 of housing 24 |
| 52 | plurality of second throughvents in replaceable portion 34 of |
| | housing 24 |
| 54 | foam pad of first apparatus 28 |
| 56 | housing facing-surface of foam pad 54 of first apparatus 28 |
| 58 | first layer of adhesive on housing facing-surface 56 of foam |
| | pad 54 of first apparatus 28 |
| 60 | housing opposing-surface of foam pad 54 of first apparatus 28 |
| 62 | second layer of adhesive on housing opposing-surface 60 of |
| | foam pad 54 of first apparatus 28 for attaching to ceiling |
| | facing-surface 22 of at least one blade 20 of ceiling fan 18 |
| 64 | peel off release-sheet of foam pad 54 of first apparatus 28 |
| 66 | pair of arms of first apparatus 28 |
| 68 | pair of hands pair of arms 66 of first apparatus 28 for |
| | releasably and compressingly engaging cage 16 of table fan 12 |
| 70 | collar of second apparatus 30 |
| 72 | shaft of second apparatus 30 |
| 74 | termination point of shaft 72 of second apparatus 30 |
| 76 | first disk of second apparatus 30 |
| 78 | center of first disk 76 of second apparatus 30 |
| 80 | plurality of third throughvents in first disk 76 of second |
| | apparatus 30 |
| 82 | second disk of second apparatus 30 |
| 84 | center of second disk 82 of second apparatus 30 |
| 85 | plurality of fourth throughvents in second disk 82 of second |
| | apparatus 30 |
| 86 | knob of second apparatus 30 |

### DETAILED DESCRIPTION OF THE PREFERRED EMBODIMENT

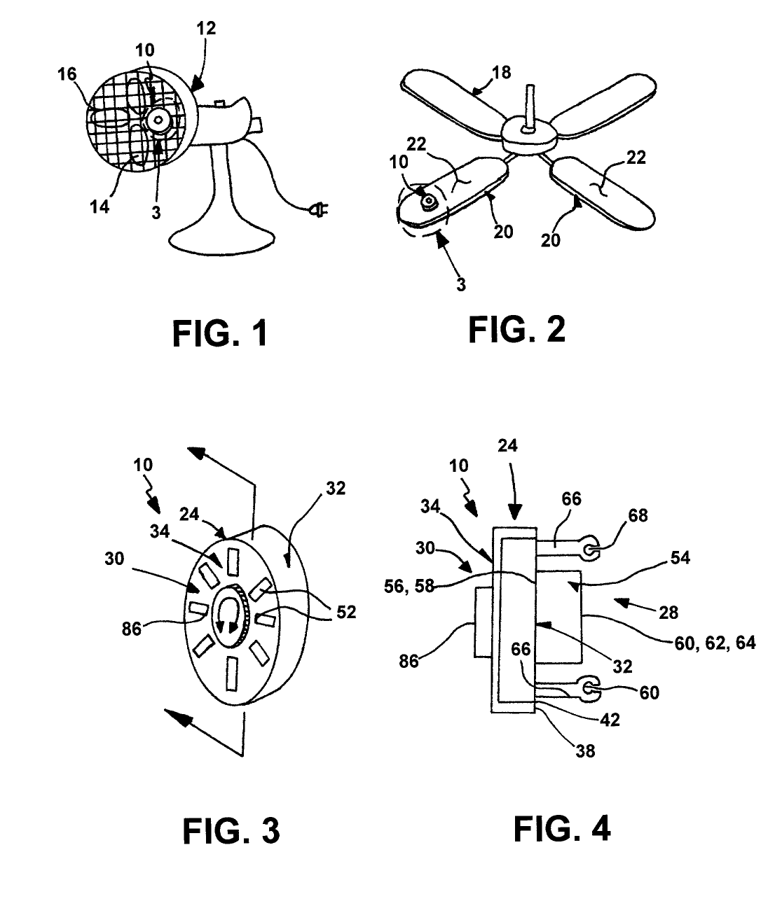

Referring now to the figures, in which like numerals indicate like parts, and particularly to FIGS. 1 and 2, which are, respectively, a diagrammatic perspective view of the present invention utilized with a table fan, and a diagrammatic perspective view of the present invention utilized with a ceiling fan, the single deodorizer of the present invention is shown generally at 10 for attaching to a variety of fans without modification, wherein the variety of fans include a table fan 12 having blades 14 and a meshed cage 16 encasing the blades 14 and a ceiling fan 18 having blades 20 with ceiling facing-surfaces 22.

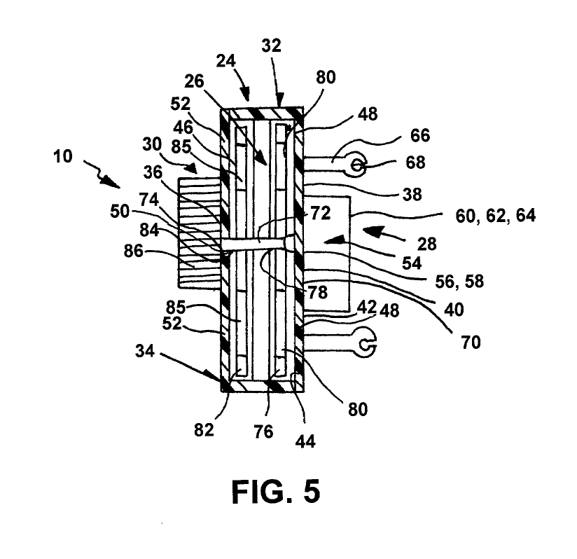

The configuration of the single deodorizer 10 can best be seen in FIGS. 3-5, which are, respectively, an enlarged diagrammatic perspective view of the area generally enclosed in the dotted curve identified by ARROW 3 in FIGS. 1 and 2 of the present invention, a diagrammatic side elevational view taken generally in the direction of ARROW 4 in FIG. 3, and an enlarged diagrammatic cross sectional view taken on LINE 5-5 in FIG. 3, and as such, will be discussed with reference thereto.

The single deodorizer 10 comprises a housing 24 for attaching to the cage 16 of the table fan 12 and to the ceiling-facing surface 22 of at least one blade 20 of the ceiling fan 18.

The single deodorizer 10 further comprises a deodorizing element 26 that is removably disposed in the housing 24 and selectively generates different amounts of a deodorant through the housing 24.

The single deodorizer 10 further comprises first apparatus 28 for attaching the housing 24 to the cage 16 of the table fan 12 and to the ceiling facing-surface 22 of the at least one blade 20 of the ceiling fan 18 without modification.

The single deodorizer 10 further comprises second apparatus 30 for selectively generating the different amounts of the deodorant through the housing 24.

The housing 24 comprises a fixed portion 32 for replaceably attaching to the cage 16 of the table fan 12 and to the ceiling-facing surface 22 of the at least one blade 20 of the ceiling fan 18, and a replaceable portion 34 that is replaceably attached to the fixed portion 32 of the housing 24, and has a center 36.

The fixed portion 32 of the housing 24 is hollow and has a rear wall 38 with a center 40, a fan-facing surface 42, and a fan-opposing surface 44, and a front wall 46 that is open.

The fixed portion 32 of the housing 24 is circular-cylindrically-shaped for reducing drag thereon when moving in a stream of fluid, as will be shown infra.

The stream function due to flow around a circular cylinder given by a doublet in a uniform horizontal stream is expressed by equation 1 infra:

$$ψ = \frac{μ}{2 Π r} sin θ - Uy$$

Equation 1 supra converted to rectangular coordinates yields equation 2 infra:

$$ψ = \frac{μ}{2 Π } \frac{y}{{x}^{2} + {y}^{2}} - Uy$$

And, for the streamline ψ=0 yields equation 3 infra:

$$y ⌊ \frac{μ}{2 Π ( {x}^{2} + {y}^{2} )} - U ⌋ - 0$$

i.e, equation 4 infra:

y=0

or equation 5 infra:

$${x}^{2} + {y}^{2} = \frac{μ}{2 Π U}$$

This shows the streamline ψ=0 to consist of Ox axis together with a circle, of center O, and of a radius a given by equation 6 infra:

$$a = \sqrt{\frac{μ}{2 Π U}}$$

Alternatively, by converting equation 1 supra to polar coordinates yields equation 7 infra:

$$ψ = \frac{μ}{2 Π r} sin θ - U r sin θ$$

Rearranging equation 7 supra yields equation 8 infra:

$$ψ = sin θ ⌊ \frac{μ}{2 Π r} - U r ⌋ = 0 for ψ = 0$$

Yielding equation 9 infra:

sin θ=0 [symbol] θ=0V±π

or yielding equation 10 infra:

$$\frac{μ}{2 Π r} - Ur = 0$$

yielding equation 11 infra:

$$r = \sqrt{\frac{μ}{2 Π U}} = a$$

The streamline ψ=0 thus consists of a circle and a straight line on a diameter produced.

Again, in this case the streamline ψ=0 separates the flow into two distinct patterns: that outside the circle coming from the undisturbed flow a long way upstream, to flow around the and again to revert to uniform flow downstream; and that inside the circle from the doublet. This is confined within the circle and does not mingle with the horizontal stream at all. This inside flow is usually neglected.

When a circular cylinder is moving in a stream of fluid, the motion around it will by given by equation 12 infra:

$$U sin θ ⌊ \frac{{a}^{2}}{r} - r ⌋$$

And, the velocity anywhere on the surface is given by equation 12 infra:

q=2U sin θ

By the use of Bernoulli's Equation, the pressure p acting on the surface of the cylinder where the velocity is q can be found.

If p₀ is the static pressure of the free stream where the velocity is U, then by Bernoulli's Equation yields equation 14 infra:

$${p}_{0} + \frac{1}{2} ρ {U}^{2} = p + \frac{1}{2} ρ {q}^{2}$$

or equation 15 infra:

$${p}_{0} + \frac{1}{2} ρ {U}^{2} = p + \frac{1}{2} {ρ ( 2 U sin θ )}^{2}$$

Therefor yielding equation 16 infra:

$$p - {p}_{0} = \frac{1}{2} ρ {U}^{2} [ 1 - 4 {sin}^{2} θ ]$$

So it can be seen that the pressure distribution is symmetrical about the axis of a circular cylinder moving in a stream, and as a result thereof, there is no drag force. See AERODYNAMICS FOR ENGINEERING STUDENTS; Houghton et al.; Edward Arnold Ltd.; 1966.

The rear wall 38 of the fixed portion 32 of the housing 24 has a plurality of first throughvents 48 that extend radially outwardly from the center 40 thereof.

The replaceable portion 34 of the housing 24 selectively and replaceably closes the front wall 46 of the fixed portion 32 of the housing 24.

The replaceable portion 34 of the housing 24 has a throughbore 50 in the center 36 thereof, and a plurality of second throughvents 52 that extend radially outwardly from the center 36 thereof, in alignment with the plurality of first throughvents 48 in the rear wall 38 of the fixed portion 32 of the housing 24.

The deodorizing element 26 is removably disposed in the fixed portion 32 of the housing 24.

The first apparatus 28 includes a foam pad 54 that has a housing facing-surface 56 with a first layer of adhesive 58 thereon so as to attach to the rear wall 38 of the fixed portion 32 of the housing 24, a housing opposing-surface 60 with a second layer of adhesive 62 thereon for attaching to the ceiling facing-surface 22 of the at least one blade 20 of the ceiling fan 18, and a peel off release-sheet 64 that overlies and protects the second layer of adhesive 62 prior to attachment.

The first apparatus 28 further includes a pair of arms 66 that extend rearwardly from the rear wall 38 of the fixed portion 32 of the housing 24, straddle the foam pad 54 of the first apparatus 28, and terminate in a pair of hands 68 for releasably and compressingly engaging the cage 16 of the table fan 12.

The second apparatus 30 includes a collar 70 that is disposed at the center 40 of the rear wall 38 of the fixed portion 32 of the housing 24, and extends inwardly from the fan-opposing surface 44 of the rear wall 38 of the fixed portion 32 of the housing 24.

The second apparatus 30 further includes a shaft 72 that extends rotatably from in the collar 70 of the second apparatus 30 rotatably through, and slightly past, the throughbore 50 in the center 36 of the replaceable portion 34 of the housing 24, to a termination point 74.

The second apparatus 30 further includes a first disk 76 that has a center 78, is disposed in the fixed portion 32 of the housing 24, between the deodorizing element 26 and the rear wall 38 of the fixed portion 32 of the housing 24, and is attached at the center 78 thereof to the shaft 72 of the second apparatus 30 for rotation therewith.

The first disk 76 of the second apparatus 30 has a plurality of third throughvents 80 that extend radially outwardly from the center 78 thereof, and which are alignable with the plurality of first throughvents 48 in the rear wall 38 of the fixed portion 32 of the housing 24.

The second apparatus 30 further includes a second disk 82 that has a center 84, is disposed in the fixed portion 32 of the housing 24, between the deodorizing element 26 and the replaceable portion 34 of the housing 24, and is attached at the center 84 thereof to the shaft 72 of the second apparatus 30 for rotation therewith.

The second disk 82 of the second apparatus 30 has a plurality of fourth throughvents 85 that extend radially outwardly from the center 84 thereof, are aligned with the plurality of third throughvents 80 in the first disk 76 of the second apparatus 30, and are alignable with the plurality of second throughvents 52 in the replaceable portion 34 of the housing 24.

The second apparatus 30 further includes a knob 86 that is disposed on the termination point 74 of the shaft 72 of the second apparatus 30 for rotation therewith.

In operation, when the knob 86 of the second apparatus 30 is turned, the shaft 72 of the second apparatus 30 turns, which causes the first disk 76 of the second apparatus 30 and the second disk 82 of the second apparatus 30 to turn, which causes a selected amount of the plurality of throughvents 85 in the second disk 82 of the second apparatus 30 to communicate with the plurality of second throughvents 52 in the replaceable portion 34 of the housing 24 and the selected amount of the plurality of throughvents 80 in the first disk 76 of the second apparatus 30 to communicate with the plurality of first throughvents 48 in the rear wall 38 of the fixed portion 32 of the housing 24, and thereby selectively generate the different amounts of the deodorant through the housing 24.

It will be understood that each of the elements described above, or two or more together, may also find a useful application in other types of constructions differing from the types described above.

While the invention has been illustrated and described as embodied in a single deodorizer for attaching to a variety of fans without modification, however, it is not limited to the details shown, since it will be understood that various omissions, modifications, substitutions and changes in the forms and details of the device illustrated and its operation can be made by those skilled in the art without departing in any way from the spirit of the present invention.

Without further analysis, the foregoing will so fully reveal the gist of the present invention that others can, by applying current knowledge, readily adapt it for various applications without omitting features that, from the standpoint of prior art, fairly constitute characteristics of the generic or specific aspects of this invention.

The invention claimed is:

1. A single deodorizer for attaching to a variety of fans without modification, wherein the variety of fans include a table fan having blades and a cage encasing the blades and a ceiling fan having blades with ceiling facing-surfaces, said deodorizer comprising:
a) a housing for attaching to the cage of the table fan and to the ceiling-facing surface of at least one blade of the ceiling fan;
b) a deodorizing element removably disposed in said housing and selectively generating different amounts of a deodorant through said housing;
c) first means for attaching said housing to the cage of the table fan and to the ceiling facing-surface of the at least one blade of the ceiling fan without modification; and
d) second means for selectively generating said different amounts of said deodorant through said housing.

2. The deodorizer as defined in claim 1, wherein said housing comprises:
a) a fixed portion for replaceably attaching to the cage of the table fan and to the ceiling-facing surface of the at least one blade of the ceiling fan; and
b) a replaceable portion that is replaceably attached to said fixed portion of said housing, and has a center.

3. The deodorizer as defined in claim 2, wherein said fixed portion of said housing is hollow, circular-cylindrically-shaped for reducing drag thereon when moving in a stream of fluid, and has:
a) a rear wall having:
i) a center;
ii) a fan-facing surface; and
iii) a fan-opposing surface; and
b) a front wall that is open.

4. The deodorizer as defined in claim 3, wherein said rear wall of said fixed portion of said housing has a plurality of first throughvents that extend radially outwardly from said center thereof.

5. The deodorizer as defined in claim 4, wherein said replaceable portion of said housing selectively and replaceably closes said front wall of said fixed portion of said housing.

6. The deodorizer as defined in claim 4, wherein said replaceable portion of said housing has:
a) a throughbore in said center thereof; and
b) a plurality of second throughvents that extend radially outwardly from said center thereof, in alignment with said plurality of first throughvents in said rear wall of said fixed portion of said housing.

7. The deodorizer as defined in claim 2, wherein said deodorizing element is removably disposed in said fixed portion of said housing.

8. The deodorizer as defined in claim 3, wherein said first means includes a foam pad that has:
a) a housing facing-surface with a first layer of adhesive thereon so as to attach to said rear wall of said fixed portion of said housing;
b) a housing opposing-surface with a second layer of adhesive thereon for attaching to the ceiling facing-surface of the at least one blade of the ceiling fan; and
c) a peel off release-sheet that overlies and protects said second layer of adhesive prior to attachment.

9. The deodorizer as defined in claim 8, wherein said first means further includes a pair of arms that extend rearwardly from said rear wall of said fixed portion of said housing, straddle said foam pad of said first means, and terminate in a pair of hands for releasably and compressingly engaging the cage of the table fan.

10. The deodorizer as defined in claim 6, wherein said second means includes a collar that is disposed at said center of said rear wall of said fixed portion of said housing, and extends inwardly from said fan-opposing surface of said rear wall of said fixed portion of said housing.

11. The deodorizer as defined in claim 10, wherein said second means further includes a shaft that extends rotatably from in said collar of said second means rotatably through, and slightly past, said throughbore in said center of said replaceable portion of said housing, to a termination point.

12. The deodorizer as defined in claim 11, wherein said second means further includes a first disk that has a center, is disposed in said fixed portion of said housing, between said deodorizing element and said rear wall of said fixed portion of said housing, and is attached at said center thereof to said shaft of said second means for rotation therewith.

13. The deodorizer as defined in claim 12, wherein said first disk of said second means has a plurality of third throughvents that extend radially outwardly from said center thereof, and which are alignable with said plurality of first throughvents in said rear wall of said fixed portion of said housing.

14. The deodorizer as defined in claim 13, wherein said second means further includes a second disk that has a center, is disposed in said fixed portion of said housing, between said deodorizing element and said replaceable portion of said housing, and is attached at said center thereof to said shaft of said second means for rotation therewith.

15. The deodorizer as defined in claim 14, wherein said second disk of said second means has a plurality of fourth throughvents that extend radially outwardly from said center thereof, are aligned with said plurality of third throughvents in said first disk of said second means, and are alignable with said plurality of second throughvents in said replaceable portion of said housing.

16. The deodorizer as defined in claim 15, wherein said second means further includes a knob that is disposed on said termination point of said shaft of said second means for rotation therewith, and when said knob of said second means is turned, said shaft of said second means turns, which causes said first disk of said second means and said second disk of said second means to turn, which causes a selected amount of said plurality of throughvents in said second disk of said second means to communicate with said plurality of second throughvents in said replaceable portion of said housing and said selected amount of said plurality of throughvents in said first disk of said second means to communicate with said plurality of first throughvents in said rear wall of said fixed portion of said housing, and thereby selectively generate said different amounts of said deodorant through said housing.
